(19) United States
(12) Plant Patent Application Publication
(10) Pub. No.: US 2003/0200586 P1
(43) Pub. Date: Oct. 23, 2003

(54) Albizia plant named 'Boubri'

(76) Inventor: M. Andre Bourquin, Aureilhan (FR)

Correspondence Address: BURNS, DOANE, SWECKER & MATHIS, L.L.P., P.O. Box 1404, Alexandria, VA, 22313-1404, US

(21) Appl. No.: 10/125,421

(22) Filed: Apr. 19, 2002

(51) Int. Cl. A01H 5/00

(52) U.S. Cl. PLT/216

(57) ABSTRACT

A new and distinct cultivar of Albizia julibrissin Durazz. is provided that is the product of a controlled breeding program that was carried out in France. An attractive spreading generally umbrella-shaped growth is displayed. Strongly fragrant blossoms are formed in profusion over an extended period of time having long tufted stamens that bear carmine magenta-red coloration of good stability. The foliage is glossy dark green and contrasts well with the blossom coloration. Good resistance to cold and no particular susceptibility to disease also are exhibited.

### BOTANICAL/COMMERCIAL CLASSIFICATION

Albizia julibrissin Durazz./Silktree

### VARIETAL DENOMINATION

cv. ‘Boubri’

### SUMMARY OF THE INVENTION

The present invention comprises a new and distinct cultivar of Albizia tree (sometimes spelled “Albizzia”), botanically known as Albizzia julibrissin Durazz., and hereafter is referred to by the cultivar name ‘Boubri’. The new cultivar is of the ‘Rosea Carr’ type.

The new cultivar of the present invention was created during 1980 at Borderes sur Léchez, France during the course of a controlled breeding program. The female parent (i.e., the seed parent) was Albizia julibrissin ‘Rosea’ (non-patented in the United States). The male parent (i.e., the pollen parent) was an unnamed Albizia julibrissin seedling (non-patented in the United States). The seeds resulting from the cross were sown and small plants were obtained which were physically and biologically different from each other. Selective study resulted in the identification of a single plant of the new cultivar of the present invention.

It was found that the new Albizia cultivar of the present invention displays:

(1) an attractive spreading generally umbrella-shaped growth habit,

(2) a profusion of attractive strongly fragrant blossoms having long tufted stamens that bear carmine magenta-red coloration of good stability,

(3) large glossy dark green foliage, and

(4) good resistance to cold.

The growth character of the new cultivar is regular with the tree growing upward in somewhat distinct stages. The hardiness is noteworthy with the tree continuing to bloom well even in the north of France.

The new cultivar well meets the needs of the horticultural industry and can provide attractive ornamentation in the landscape as a distinctive specimen plant.

Asexual reproduction of the new cultivar by the use of cuttings as performed at Borderes sur Léchez, France, in a controlled environment has demonstrated that the characteristics of the new cultivar are firmly fixed and are retained through successive generations of asexual propagation.

‘Boubri’ has not been observed under all possible environmental conditions to date. Accordingly, it is possible that the phenotype may vary somewhat with variations in the environment, such as temperature, light, day length, etc.

### BRIEF DESCRIPTION OF THE PHOTOGRAPHS

The depicted trees of the ‘Boubri’ cultivar were reproduced by the rooting of cuttings and were grown outdoors in the landscape at Borderes sur Léchez, France.

FIG. 1 illustrates during the summer a typical specimen of a five year-old tree. A profusion of the attractive carmine magenta-red blossoms, and dark green foliage are apparent.

FIG. 2 illustrates during the summer a typical specimen of a twelve year-old tree. The spreading generally umbrella-shaped growth habit, attractive carmine magenta-red blossoms, and dark green foliage similarly are apparent.

FIG. 3 illustrates a closer view of typical inflorescence and foliage.

FIG. 4 illustrates typical inflorescence wherein the colorful long tufted stamens are shown. The attractive glossy dark green foliage also is visible.

### DETAILED DESCRIPTION

The plant described was a sixteen year-old specimen of the ‘Boubri’ cultivar while growing outdoors in the landscape at Borderes sur Léchez, France. The tree was observed from mid-July to the end of September. The color terminology utilized in the description is to be accorded its ordinary dictionary significance.

Plant:

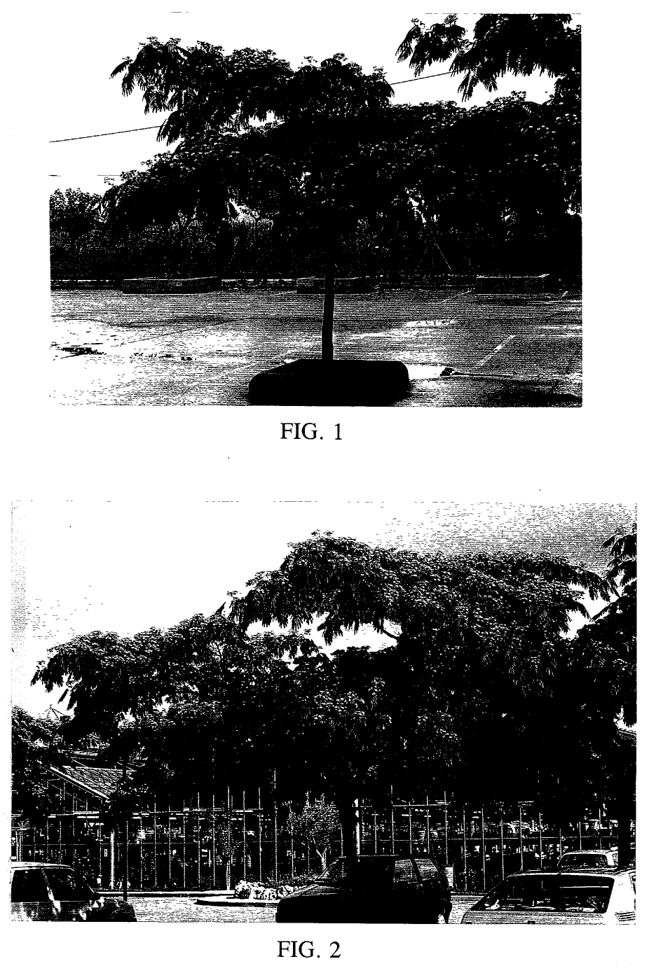

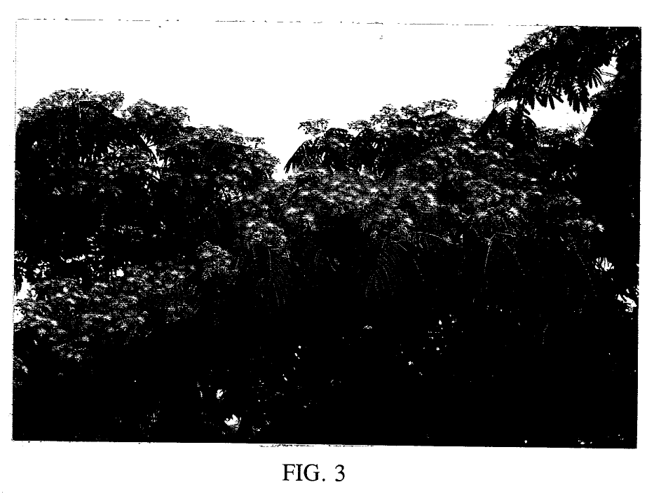

Form.—The crown displays an attractive spreading generally umbrella-shaped growth habit. As illustrated in FIGS. 1 and 2, there is significant horizontal spreading. The tree grows upward in somewhat distinct stages or horizontal ramifications.

Height.—Up to approximately 12 meters when fully mature.

Branches:

Young stems.—dark greenish-gray and striped with dark brown.

Adult wood.—gray on a greenish surface. The lenticels are whitish.

Foliage:

Shape.—deciduous leaves are arranged bipinnate in pairs of approximately 10 to 12 pennules on average which divide into approximately 20 to 30 pairs of leaflets.

Length.—approximately 25 cm on average.

Width.—approximately 12 cm on average.

Petiole.—approximately 3 to 5 cm in length on average. The upper surface is greenish-yellow and the under surface is pale green.

Rachis.—bears a gland at the base.

Leaflets.—number: approximately 200 to 360 per leaf. — shape: curved-oblong, falciform and frondlike. — texture: glossy and smooth on the upper and under surface. — general appearance: finely textured and tropical in appearance. — new foliage color: glossy dark green and sometime purple on the upper surface and pale green on the under surface. — mature foliage color: glossy dark green on the upper surface and greenish cream on the under surface.

Inflorescence:

Blooming period.—substantially continuous from mid-July to the end of September.

Bearing.—in pedunculate clusters of approximately 20 to 30 on average forming a corymb panicle having a length of approximately 3 to 4 cm. The clusters flower from the bottom to the top.

Buds.—diameter: approximately 1.5 cm on average. — shape: round. — color: cream.

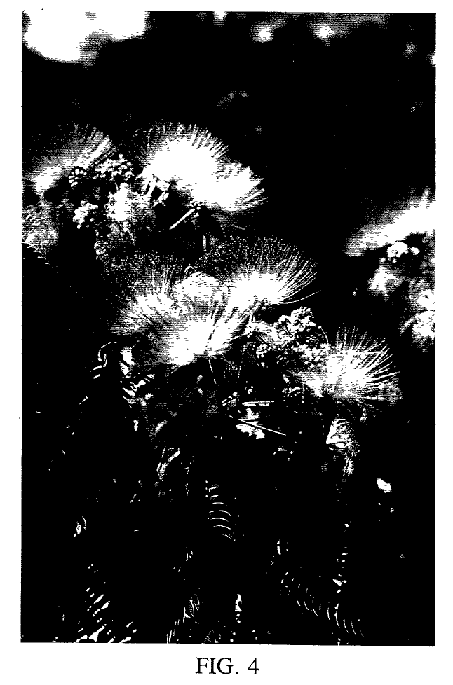

Flowers.—calyx and corolla: cream green in coloration. — stamen shape: form a feathery tuft. — stamen length: approximately 3.5 to 4 cm on average. — stamen number: commonly in excess of 40. — filaments: provide color to the stamens and are cream colored at the base and carmine magenta-red toward the tip as illustrated in FIG. 4. — stamen texture: very smooth. — fragrance: very strong and similar to that of the wood of lime. — anthers: minuscule in size and yellow in coloration. — pollen: present and yellow in coloration. — fruit: generally flattened and in the shape of an elongated pod with a wrinkled surface. The coloration is creamy-white. The length is approximately 15 to 18 cm on average and the width is approximately 2.5 to 3 cm on average. — seeds: there commonly are approximately 10 to 15 seeds per fruit on average. The seed configuration is lenticular oval and the coloration is brown.

Tolerance to Diseases: Very good with no particular susceptibility to diseases having been observed during observations to date.

Hardiness: Very good. The ‘Boubri’ cultivar has been found to well resist cold and continues to blossom even when tested in the north of France.

I claim:

1. A new and distinct cultivar of Albizia julibrissi Durazz. that displays the following combination of characteristics:
(a) an attractive spreading generally umbrella-shaped growth habit,
(b) a profusion of attractive strongly fragrant blossoms having long tufted stamens that bear carmine magenta-red coloration of good stability,
(c) large glossy dark green foliage, and
(d) good resistance to cold;
substantially as illustrated and described.
